(19) United States
(12) Patent Application Publication
(10) Pub. No.: US 2005/0281889 A1
(43) Pub. Date: Dec. 22, 2005

(54) Nutritional supplement for adults

(76) Inventor: Ranjit Kumar Chandra, Gurgaon (IN)

Correspondence Address: Thomas P. Liniak;Liniak, Berenato & White, Suite 240, 6550 Rock Spring Drive, Bethesda, MD, 20817, US

(21) Appl. No.: 10/867,711

(22) Filed: Jun. 16, 2004

(51) Int. Cl. A61K 31/525; A61K 31/51; A61K 31/355

(52) U.S. Cl. 424/604; 424/638; 424/641; 424/702; 424/646; 424/639; 514/251; 514/276; 514/350; 514/356; 514/168; 514/474; 514/458; 514/725

(57) ABSTRACT

A multinutrient nutritional supplement is provided that is designed to be most effective in optimizing health, increasing the immunity and decreasing the instances and severity of infection particularly among adults.

### BACKGROUND OF THE INVENTION

1. Field of the Invention

The present invention relates to nutritional supplements, and more particularly relates to nutritional supplements optimized for administration to adults. The invention further relates to nutritional supplements optimized for enhanced immunological response in adults.

2. Description of the Related Art

In the United States, Canada, Western Europe, Japan, and other industrialized affluent countries, our exposure to “malnutrition” comes from the print and audiovisual media, and visual images in newspapers, magazines, and television. These media highlight the problem of nutritional deficiencies that are widely observed in less developed countries and in underprivileged populations in general. In addition to protein-energy malnutrition, deficiencies of individual nutrients are widespread throughout the world. In particular, deficiencies of iron, vitamin A and iodine are endemic in some regions in all continents. It has been estimated that at least 600 million individuals suffer from such deficiencies.

It is surprising that nutrient deficiencies may also be seen in particular age groups in the United States, Canada, and other affluent countries. Iron deficiency was very common in adults and young children but the advent of iron-fortified adult formulas and of fortified cereals has reduced its incidence dramatically. However, these problems have not disappeared.

Nutritional deficiencies occurring in adults are many and vary according to geographic setting and socioeconomic status of the individual. Even though such deficiencies are less common among adults 20-50 compared to the elderly, these deficiencies do occur even in industrialized countries such as the United States. Poverty and lack of nutrition knowledge and health education among individuals compound the problem. Factors other poverty can lead to nutrient deficiencies in adults ages 20 to 50. Many adults lead very busy lives and do not eat a balanced diet. This life style often leads to missed meals, especially breakfast, and unbalanced lunches. Although some foods sold in the market are fortified with a few nutrients, this is not a systematic fortification, leaving grounds for deficiencies. Furthermore, common illnesses and infections to adults, such as respiratory infections, and physiological states such as menstruation in females worsen the risk of nutritional deficiencies.

It is now recognized that certain age groups and some physiological states require additional amounts of nutrients. A case in point is pregnancy. Pregnant women are recognized to need a greater amount of various nutrients not only to maintain their own good health but also ensure an adequate growth and satisfactory state of the unborn baby. These needs have been addressed, for example, in U.S. Pat. No. 4,994,283 directed to nutritional mineral supplements which include iron and calcium compounds in combination with citrates or tartrates, ascorbates, and fructose in an effort to reduce the tendency of calcium to inhibit the bioavailability of iron, so that the conjoint bioavailability of these two minerals is enhanced. Another case in point is childhood years of 4-12 years of age. The needs of this age class are outlined and addressed, for example, in U.S. Pat. No. 6,565,891.

Another group with special nutrient needs is the elderly. Their numbers are increasing and their health needs are much greater than those of younger individuals because they are ill more often and each episode of illness, such as pneumonia or fracture, requires a longer time to recover from. It has been estimated that at least one-third of the elderly in the US and Canada and Europe have objective evidence of nutritional deficiencies. The art described in U.S. Pat. No. 5,556,644 addressed these concerns and special needs, and is hereby incorporated by reference.

The special nutrient needs of adults are recognized. This is consequent on their rapid growth and increased needs of vitamins and minerals for various physiological systems. Although adult nutritional supplements are available commercially, the amounts of nutrients contained in the supplements are generally arbitrary and lack a scientific or experimental basis.

### SUMMARY OF THE INVENTION

It is an object of the present invention to overcome the shortcomings of preparations that contain single nutrients or a combination of a limited number of nutrients.

It is another object of the invention to provide a nutrient supplement specifically tailored for administration to adults of 20 to 50 years of age to meet their special needs, and to provide a method for maintaining the optimal health and immunological function of adults by administration of the same. The formulation of embodiments of the invention described herein has been found to benefit vitamin and mineral supplementation for adults and at the same time minimize undesirable side effects that accrue from the administration of single nutrients and large doses of nutrients.

It is another object of the invention to provide a composition and method uniquely optimized to sustain physical and mental health of adults age 20 to 50 years, and in particular to maximize their immunological function.

Yet another object of the present invention is to provide an improved multinutrient supplement for adults that promotes higher numbers of selected T cell subsets and natural killer cells, enhanced lymphocyte proliferation, enhanced proliferation response to mitogen, increased interleukin-2 production, and higher antibody response and natural killer cell activity.

To achieve one or more of the foregoing objects, the invention according to an aspect of the invention provides a multivitamin, mineral and trace element supplement for administration to adults age 20 to 50 years, which comprises phosphorus in the amount of about 85 to about 115 mg (milligrams); chromium in the amount of about 51 to about 69 μg (mcg or micrograms); copper in the amount of about 1.7 to about 2.3 mg; iodine in the amount of about 85 to about 115 μg; iron in the amount of about 9.4 to about 12.6 mg; manganese in the amount of about 0.85 to about 1.15 mg; molybdenum in the amount of about 34 to about 46 μg; selenium in the amount of about 170 to about 230 μg; zinc in the amount of about 10.2 to about 13.8 mg; vitamin A in the amount of about 544 to about 736 μg; vitamin C in the amount of about 51 to about 69 mg; vitamin D in the amount of about 3.4 to about 4.6 μg; vitamin E in the amount of about 8.5 to about 11.5 mg; vitamin K in the amount of about 17 to about 23 μg; vitamin B₁ in the amount of about 0.94 to about 1.26 mg; vitamin B₂ in the amount of about 1.19 to about 1.61 mg; niacin in the amount of about 13.6 to about 18.4 mg; vitamin B₆ in the amount of about 1.7 to about 2.3 mg; folic acid in the amount of about 170 to about 230 μg; vitamin B₁₂ in the amount of about 0.68 to about 0.92 μg; pantothenic acid in the amount of about 3.4 to about 4.6 mg; and biotin in the amount of about 120 to about 160 μg. Preferably yet optionally, the supplement of this aspect of the invention further comprises calcium in the amount of about 85 to about 115 mg and/or magnesium in the amount of about 85 to about 115 mg.

An advantage of embodiments of the present invention is that the nutritional supplement of the embodiments supplies the right amount of the necessary nutrients including vitamins and minerals to adults to assure optimal intake of nutrients needed for health and maximal immunological response and protection against nutritional losses and deficiencies due to lifestyle factors and inadequate dietary patterns.

Another advantage of embodiments of the present invention is that the nutritional supplement of the embodiments provides the necessary vitamins and minerals to allow adults using the supplement to maintain their present health and positively influence their future health.

Another advantage of embodiments of the present invention is that the nutritional supplement of the embodiments increases and/or optimizes the immunological responses of adults that are users of the supplement, such immunological responses including lymphocyte response to PHA, interleukin-2, antibody response and thymulin activity.

Still another advantage of embodiments of the present invention is that the nutritional supplement of the embodiments reduces the occurrence of common infections in the adults that are using the supplement.

These and other advantages and benefits of the present invention will be apparent to those skilled in the art upon reading and understanding the following detailed description of the invention.

### BRIEF DESCRIPTION OF THE FIGURE

The accompanying drawing is incorporated in and constitutes a part of the specification. The drawing, together with the general description given above and the detailed description of the preferred embodiments and methods given below, serves to explain the principles of the invention. In such drawing:

FIG. 1 is a dose response graph illustrating lymphocyte response to mitogen based on zinc amounts.

### DETAILED DESCRIPTION OF THE PREFERRED EMBODIMENTS

The present invention as embodied in certain embodiments described herein is directed to a nutritional supplement containing the appropriate amounts of vitamins and trace elements that sustains an optimum level of immunity and reduces the incidence of infections among 20 to 50 year old adults, both males and females. As noted above, none of the nutrient preparations in the market have documented benefits shown by this invention.

A first embodiment of the present invention is directed to a nutritional supplement for administration to adults to enhance and improve their immunological response which comprises phosphorus in the amount of about 85 to about 115 mg; chromium in the amount of about 51 to about 69 μg; copper in the amount of about 1.7 to about 2.3 mg; iodine in the amount of about 85 to about 115 μg; iron in the amount of about 9.4 to about 12.6 mg; manganese in the amount of about 0.85 to about 1.15 mg; molybdenum in the amount of about 34 to about 46 μg; selenium in the amount of about 170 to about 230 μg; zinc in the amount of about 10.2 to about 13.8 mg; vitamin A in the amount of about 544 to about 736 μg; vitamin C in the amount of about 51 to about 69 mg; vitamin D in the amount of about 3.4 to about 4.6 μg; vitamin E in the amount of about 8.5 to about 11.5 mg; vitamin K in the amount of about 17 to about 23 μg; vitamin B₁ in the amount of about 0.94 to about 1.26 mg; vitamin B₂ in the amount of about 1.19 to about 1.61 mg; niacin in the amount of about 13.6 to about 18.4 mg; vitamin B₆ in the amount of about 1.7 to about 2.3 mg; folic acid in the amount of about 170 to about 230 μg; vitamin B₁₂ in the amount of about 0.68 to about 0.92 μg; pantothenic acid in the amount of about 3.4 to about 4.6 mg; and biotin in the amount of about 120 to about 160 μg. Preferably yet optionally, the supplement of this first embodiment of the invention further comprises calcium in the amount of about 85 to about 115 mg and/or magnesium in the amount of about 85 to about 115 mg.

The adult's nutritional supplement of a second embodiment of the present invention is more particularly directed to a supplement comprising phosphorus in the amount of about 100 mg; chromium in the amount of about 60 μg; copper in the amount of about 2 mg; iodine in the amount of about 100 μg; iron in the amount of about 11 mg; manganese in the amount of about 1 mg; molybdenum in the amount of about 40 μg; selenium in the amount of about 200 μg; zinc in the amount of about 12 mg; vitamin A in the amount of about 640 μg; vitamin C in the amount of about 60 mg; vitamin D in the amount of about 4 μg; vitamin E in the amount of about 19 mg; vitamin K in the amount of about 20 μg; vitamin B₁ in the amount of about 1.1 mg; vitamin B₂ in the amount of about 1.4 mg; niacin in the amount of about 16 mg; vitamin B₆ in the amount of about 2 mg; folic acid in the amount of about 200 μg; vitamin B₁₂ in the amount of about 0.8 μg; pantothenic acid in the amount of about 4 mg; and biotin in the amount of about 140 μg. Preferably yet optionally, the adult's nutritional supplement of this second embodiment further comprises calcium in the amount of about 100 mg and/or magnesium in the amount of about 100 mg.

An adult nutritional supplement according to a third embodiment of the invention comprises, as components, phosphorus in the amount of about 85 to about 115 mg; chromium in the amount of about 51 to about 69 μg; copper in the amount of about 1.7 to about 2.3 mg; iodine in the amount of about 85 to about 115 μg; iron in the amount of about 9.4 to about 12.6 mg; manganese in the amount of about 0.85 to about 1.15 mg; molybdenum in the amount of about 34 to about 46 μg; selenium in the amount of about 170 to about 230 μg; zinc in the amount of about 10.2 to about 13.8 mg; vitamin A in the amount of about 544 to about 736 μg; vitamin C in the amount of about 51 to about 69 mg; vitamin D in the amount of about 3.4 to about 4.6 μg; vitamin E in the amount of about 8.5 to about 11.5 mg; vitamin K in the amount of about 17 to about 23 μg; vitamin B₁ in the amount of about 0.94 to about 1.26 mg; vitamin B₂ in the amount of about 1.19 to about 1.61 mg; niacin in the amount of about 13.6 to about 18.4 mg; vitamin B₆ in the amount of about 1.7 to about 2.3 mg; folic acid in the amount of about 170 to about 230 μg; vitamin B₁₂ in the amount of about 0.68 to about 0.92 μg; pantothenic acid in the amount of about 3.4 to about 4.6 mg; and biotin in the amount of about 120 to about 160 μg, wherein any one or more of the components is more specifically present in a concentration as follows: phosphorus in the amount of about 100 mg; chromium in the amount of about 60 μg; copper in the amount of about 2 mg; iodine in the amount of about 100 μg; iron in the amount of about 11 mg; manganese in the amount of about 1 mg; molybdenum in the amount of about 40 μg; selenium in the amount of about 200 μg; zinc in the amount of about 12 mg; vitamin A in the amount of about 640 μg; vitamin C in the amount of about 60 mg; vitamin D in the amount of about 4 μg; vitamin E in the amount of about 19 mg; vitamin K in the amount of about 20 μg; vitamin B₁ in the amount of about 1.1 mg; vitamin B₂ in the amount of about 1.4 mg; niacin in the amount of about 16 mg; vitamin B₆ in the amount of about 2 mg; folic acid in the amount of about 200 μg; vitamin B₂ in the amount of about 0.8 μg; pantothenic acid in the amount of about 4 mg; and/or biotin in the amount of about 140 μg.

Preferably yet optionally, the supplement of this third embodiment further comprises calcium in the amount of about 85 to about 115 mg (or about 100 mg) and/or magnesium in the amount of about 85 to about 115 mg (or about 100 mg).

Each of the component vitamins and minerals making up the nutritional supplement of the present invention are preferably provided in bioavailable forms. This means that absorption and utilization are enhanced. As a result, more of the nutrients provided will actually be available to the adult user, rather than simply passing through the digestive track unused by the body. However, other forms of the components may be used if the amounts of each component are adjusted to give similar bioavailable quantities.

Calcium

Calcium is the most common mineral in the human body and is vitally important because adequate intakes are an important determinant of bone health and risk of fracture or osteoporosis. Calcium has four major biological functions: 1) structural as stores in the skeleton, 2) electrophysiological—carries charge during an action potential across membranes, 3) intracellular regulator, and 4) as a cofactor for extracellular enzymes and regulatory proteins. Although acute deficiency symptoms are avoided because of the large skeletal stores, prolonged bone resorption from chronic dietary deficiency can lead later in life to osteoporosis due to inadequate accumulation of bone mass during growth in adolescence. Dietary calcium deficiency also has been associated with increased risk of hypertension, preeclampsia, and colon cancer. Increasing calcium intakes during adolescence increases calcium accretion up to 1300 mg/day and increases bone mineral content.

Chromium

Chromium is an essential nutrient required for normal sugar and fat metabolism and functions primarily by potentiating the action of insulin. Signs of deficiency include impaired glucose tolerance and elevated circulating insulin. In some studies, chromium supplementation has reduced total serum cholesterol, triglycerides and apolipoprotein B and increased HDL-cholesterol.

Copper

Copper is an essential trace element involved in the absorption, storage and metabolism of iron. Copper deficiency is often observed in those suffering from malnutrition and can result in anemia, cardiac abnormalities such as blood vessel and heart rupture, abnormal EKG's and elevated levels of serum cholesterol, triglycerides and glucose. A lifetime of marginal diet copper is thought to lead to heart disease.

Copper helps keep blood vessels elastic, is needed for the formation of elastin and collagen, functions as an iron oxidizer, and is needed for the proper functioning of vitamin C. In a preferred embodiment of the invention, copper is dosed in a pharmaceutically acceptable copper compound including, but not limited to, cupric oxide, cupric sulfate, cupric gluconate, and combinations thereof.

Iodine

Iodine forms an essential component of the thyroid hormones which regulate cell activity and growth in virtually all tissues and are, therefore, essential for both normal growth and development. Iodine deficiency impairs growth and neurological development, which can damage the brain and can lead to a wide spectrum of health problems, ranging from mild intellectual impairment to severe mental retardation, growth stunting, apathy, and impaired movement, speech or hearing. Cretinism is a rare disorder in which many of these abnormalities occur, represents the extreme of early iodine deficiency. Much more widespread is an intellectual blunting that may afflict as many as 50 million of the estimated 1.6 billion “at-risk” people living in iodine deficient regions, making iodine deficiency the most common preventable cause of mental retardation in the world. Because of decreased production of thyroid hormones, iodine deficiency causes compensatory hypertrophy of the thyroid gland as it attempts to make more thyroid hormone, resulting in a goiter—a disfiguring condition that is common in high-risk areas. Collectively, health problems arising from a lack of iodine are known as iodine deficiency disorders (IDD).

Iron

Iron is an essential nutrient that carries oxygen and forms part of the oxygen-carrying proteins, hemoglobin in red blood cells and myoglobin in muscle. It is also a necessary component of various enzymes. Iron deficiencies result in anemia.

Any pharmaceutically acceptable iron compound can be used in the nutritional supplement of the present invention and may be chosen from any of the well-known iron II (ferrous) or iron III (ferric) supplements, such as ferrous sulfate, ferric chloride, ferrous gluconate, ferrous lactate, ferrous tartrate, iron-sugar-carboxylate complexes, ferrous fumarate, ferrous succinate, ferrous glutamate, ferrous citrate, ferrous pyrophosphate, ferrous cholinisocitrate, ferrous carbonate, and the like.

In a further embodiment of the present invention, the iron compound comprises a pharmaceutically acceptable ferrous sulfate compound coated with a pharmaceutically acceptable film forming material which permits release of the ferrous sulfate in the intestine of the adolescent administered the supplement. Suitable coatings include any material known in the art for forming enteric, controlled release, or sustained release coatings, such as cellulose ethers including hydroxypropyl methylcellulose, methylcellulose, ethylcellulose, and carboxymethylcellulose; cellulose esters such as cellulose acetate, cellulose acetate phthalate, and cellulose nitrate; acrylate and methacrylate copolymers; and the like. The coated iron compound has been found to provide increased iron bioavailability by minimizing interaction between the iron compound and divalent cations such as calcium in the nutritional supplement. In addition, the coated iron compound is better tolerated and causes few stomach problems.

Magnesium

Magnesium plays a central role in the secretion and action of insulin and in glucose metabolism. The mineral helps control blood sugar and is able to prevent many diabetic complications. Magnesium has an essential role in the proper functioning of the entire cardiovascular system. Magnesium deficiency can be a predisposition to many conditions such as heart disease, kidney stones, cancer, and insomnia. Deficiency is common enough in the United States that scientists have recommended fortifying drinking water with magnesium to the Federal Drug Administration. Any pharmaceutically acceptable magnesium compound can be used.

Manganese

Manganese is important to maintain the integrity of the skin, bone and menstrual cycle, and in cholesterol metabolism. Any pharmaceutically acceptable manganese compound can be used.

Molybdenum

Molybdenum is an essential nutrient that is a component of a number of enzymes involved in the metabolic process.

Phosphorus

Phosphorus is an essential mineral that is found in all cells within the body. The metabolism of all major metabolic substrates depends on the functioning of phosphorus as a cofactor in a variety of enzymes and as the principal reservoir for metabolic energy.

Selenium

Selenium is an essential trace element that functions as a component of enzymes involved in antioxidant protection and thyroid hormone metabolism. Characteristic signs of selenium deficiency have not been described in humans, but very low selenium status is a factor in the etiologies of a juvenile cardiomyopathy (Keshan Disease) and a chondrodystrophy (Kashin-Beck Disease) that occur in selenium-deficient regions of China.

Zinc

Zinc is required for proper formation of DNA and RNA and is needed for growth and sexual development of young women and men, including children and adolescents. Deficiency signs include skin rashes, poor discolored hair, diarrhea, respiratory infections, and poor taste.

Vitamin A

Vitamin A is a fat-soluble vitamin. The best-known function of vitamin A is in vision, where it participates in the visual cycle. Night blindness is one of the early signs of vitamin A deficiency, because of the role of vitamin A in vision. Bacterial invasion and permanent scarring of the cornea of the eye (xerophthalmia) is a symptom of more profound deficiency. Profound vitamin A deficiency also results in altered appearance and function of skin, lung, and intestinal tissues. Adults and children are most at risk of vitamin A deficiency because they have not yet developed adequate vitamin A stores. It has been estimated that 5 million children in the world become blind each year, 70% of these due to vitamin A deficiency. Over half of these blind children die from malnutrition and associated illnesses.

Vitamin D

Vitamin D assists in the mineralization and calcification of bone, prevents rickets in children, prevents osteomalacia in adults, preserves bone and tooth growth, and lowers blood pressure. Vitamin D is fat-soluble.

Vitamin E

Vitamin E is also fat-soluble and is needed for the maintenance of cell membranes and for neurological health. Vitamin E is the generic term for a group of related substances which include alpha-tocopherol, beta-tocopherol, gamma-tocopherol, and delta-tocopherol. In addition, each of these four compounds has a “d” form, which is the natural form, and a “d1” form, which is the synthetic form.

Vitamin K

Vitamin K is a fat-soluble nutrient important for optimum functioning of the coagulation system. Since the coagulation system, hormonal system, and the immune system interact with one another, Vitamin K plays a role in ensuring optimum immunity and resistance to infection.

Thiamin

Thiamin (vitamin B-1) is a water-soluble substance, consisting of thiazole and pyrimidine rings joined by a methylene bridge and has a biologic half-life in the body of about 15 days. Thus thiamin-deficient diets will rapidly show effects of thiamin deficiencies. Deficiency signs include neuralgias and swellings of the face and legs.

Riboflavin

Riboflavin (vitamin B-2) participates in oxidation-reduction reactions in numerous metabolic pathways and in energy production via the respiratory chain. Riboflavin is used therapeutically to ameliorate ariboflavinosis resulting from diverse causes such as inadequate dietary intake, decreased assimilation, rare genetic defects in the formation of specific flavoproteins, hormonal disorders and after use of certain drugs. Deficiency signs include rough skin, angular stomatitis, cracked lips, and mouth ulcers.

Niacin

Niacin (nicotinic acid or nicotinamide) is essential in the form of the coenzymes NAD and NADP in which the nicotinamide moiety acts as electron acceptor or hydrogen donor in many biological redox reactions.

Pellagra, the classic niacin deficiency disease, is characterized by symmetrical dermatitis, diarrhea, and dementia. Often associated with a largely cereal diet such as maize or sorghum, the disease is now rarely seen in industrialized countries but still appears in India, China, and Africa. Pellagra often is associated with other micronutrient deficiencies and may develop also in cases of disturbed tryptophan metabolism (carcinoid syndrome, Hartnup's).

Vitamin B6

Vitamin B6 or pyridoxine is involved in the production of ribonucleic acid (RNA) and deoxyribonucleic acid (DNA) and many other reactions in the body. Pyridoxine refers to and includes three different compounds: pyridoxine, pyridoxamine, and pyridoxal. hydrochloride. Deficiency or dependency has been associated with diarrhea, rough skin, anemia and convulsions.

Folate

Folate is an essential vitamin that plays a role in the synthesis of RNA, DNA and protein, and thus the folate requirement and, consequently, the risk of deficiency is elevated during periods of rapid growth such as in adolescence. Low folate intakes also are correlated with high levels of serum homocysteine which are associated with an increased risk of atherosclerosis and several forms of vascular disease. Folate is present in many foods, however, the folate content of foods is inherently variable and a large fraction of the folate consumed each day comes from foods that are frequently ingested, but not particularly concentrated, sources of the vitamin. Flour sold in some countries such as USA and Canada is fortified with folic acid.

Vitamin B12

Vitamin B12 or the cobalamins is necessary for overall metabolism, the function of the nervous system, metabolism of folic acid, and the production of red blood cells. There are at least three active forms of cobalamin: cyanocobalamin, hydroxocobalamin, and nitrocobalamin. In a preferred embodiment of the present invention, vitamin B12 is provided in the form of cyanocobalamin. Deficiency or dependency has been associated with diarrhea, rough skin, anemia and convulsions.

Pantothenic Acid

Pantothenic acid is important for the production of adrenal gland hormones, increases overall energy, and helps convert food into energy.

Biotin

Biotin, also known as vitamin H and coenzyme R (Hexahydro-2-oxo-1H-thienal[3,4-d]-imidazole-4-pentatonic acid), functions as an essential cofactor for four carboxylases that catalyze the incorporation of cellular bicarbonate into the carbon backbone of organic compounds. Severe deficiencies of biotin can cause thinning of hair, loss of hair color, and eventually complete loss of hair; a scaly, red rash distributed around the openings of the eyes, nose, mouth, and perineal area; and central nervous system abnormalities such as depression, lethargy, hallucinations, and paresthesias.

Administration

The nutritional supplements of the invention may be provided in any suitable dosage form known in the art. For example, the compositions may be incorporated into tablets, powders, granules, beads, chewable lozenges, capsules, aqueous suspensions or solutions, other liquid forms, or similar conventional dosage forms, using conventional equipment and techniques known in the art. When preparing dosage forms incorporating the compositions of the invention, the nutritional components are normally blended with conventional excipients such as binders, including gelatin, pregelatinized starch, and the like; lubricants, such as hydrogenated vegetable oil, stearic acid, and the like; diluents, such as lactose, mannose, and sucrose; disintegrants, such as carboxymethyl cellulose and sodium starch glycolate; suspending agents, such as povidone, polyvinyl alcohol and the like; absorbents, such as silicon dioxide; preservatives, such as methylparaben, propylparaben, and sodium benzoate; surfactants, such as sodium lauryl sulfate, polysorbate 80, and the like; and colorants, such as F.D. & C dyes and the like. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening and/or flavoring and/or coloring agents.

All of the aforementioned benefits are achieved without wasting vitamin and mineral materials, as characteristic of unitary supplements of the prior art and without the detriment of an excess of some or all of the vitamin and mineral materials. This makes the products of the invention not only more cost effective than conventional supplements, but also, and more significantly, without the detriment of an overdose of any vitamin or mineral materials.

Although the products of the invention are preferably intended for administration to humans, it will be understood that the formulations may also be utilized in veterinary therapies for other animals.

The following example is given to illustrate the invention but is not deemed to be limiting thereof. All amounts specified in the application are based on milligrams unless otherwise indicated.

### EXAMPLES

### Example 1

### Dose Response Graphs

The basic concept underlying the assessment of the most optimum nutrient amounts for a given age group uses the principle of “dose-response graphs”. At least four groups of individuals were provided with various amounts of a given nutrient and their immune responses were measured using established and accepted techniques. The amount of a nutrient that gave the best response was considered the optimum amount. Dose response graphs were determined for all vitamins and trace elements considered essential for human health, particularly immunity.

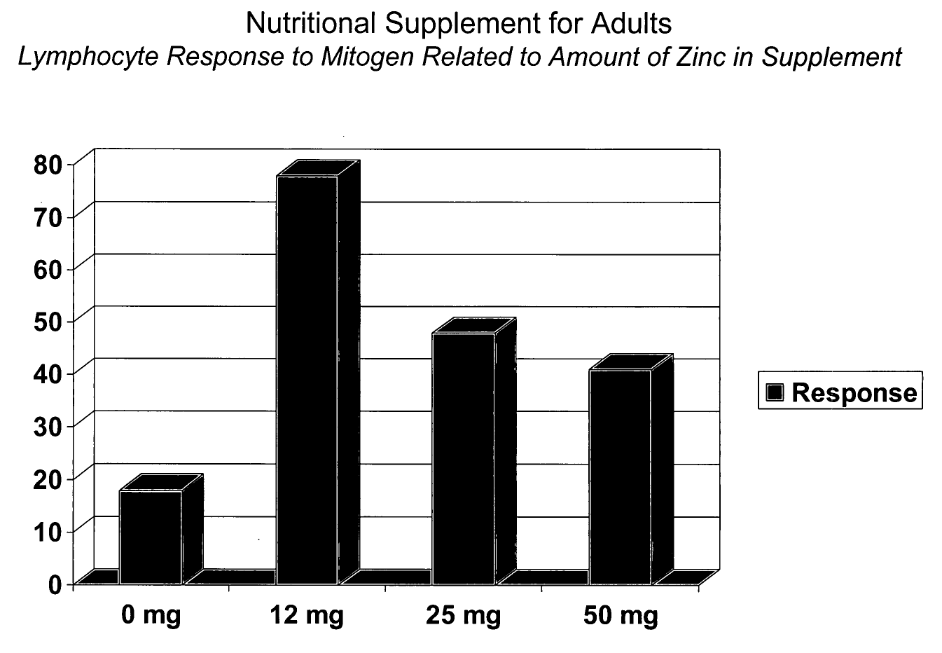

Data for the dose response chart for zinc in a group of 20 to 50 year old adults is shown in FIG. 1, with the total amount of zinc taken is shown for each group. To determine the magnitude of immune response, an aliquot of blood was withdrawn from each subject in the study, blood lymphocytes were separated, washed and mixed with the mitogen phytohemagglutinin (PHA) in previously determined optimum amount. The optimum amount of PHA used in the experiments was predetermined by a set of dose response graphs using lymphocytes of a healthy donor and four different concentrations of PHA. After culture in a sterile environment for 72 hours, the cells were washed and mixed with radioactive thymidine. The same steps were undertaken in a control sample in which only the culture medium was used, not PHA. The cells were washed and radioactivity determined.

TABLE 1
Effect of various amounts of zinc on immune responses in adults, age
20-50 years.

| | Amount of supplemental zinc (mg) | | | |
|---|---|---|---|---|
| | 0 | 12 | 25 | 50 |
| Lymphocyte response | 18 | 78 | 48 | 41 |
| to PHA (stimulation | | | | |
| index) | | | | |

It was concluded from this study that 12 mg of zinc intake, taken in combination with the amount of zinc an adult intakes from a balanced diet, produced the best immune response. Dietary intake of zinc was calculated based on three 24-hour recalls and by the food frequency questionnaire methods.

### Example 2

### Randomized Controlled Trial

The most widely accepted ideal method of showing the positive or negative benefits of a treatment modality is the randomized controlled trial (RCT). It can be further refined and made more objective by using the principles of double-blinded observations and placebo-controlled. This implies that a group of study subjects are recruited for the trial. Based on computer-generated random numbers, each individual is assigned to one of the two study groups: “Experimental” who receives the study product, “placebo” who receives the inert or dummy product.

The subjects are observed both clinically and their blood samples are tested periodically. Infection is diagnosed on clinical grounds as also by appropriate laboratory tests on blood, urine, sputum, and by radiographs of the chest, sinuses or other regions, as deemed appropriate for the individual at that time.

The “Experimental” group received the study product, which was made up by mixing together the nutrients in amounts shown in Table 2. The results of the randomized prospective double-blind placebo-controlled trial conducted over a period of 12 months were used to draw conclusions.

From these experiments, it was determined that the supplemental amounts of various vitamins and trace elements, other than what was present in the average diet, that gave the maximum immune response in a group of 20 to 50 year old adults was as set forth in Table 2.

TABLE 2
Optimized supplement formulation for adults

| | | |
|---|---|---|
| Calcium | 100 | mg |
| Chromium | 60 | ug (mcg) |
| Copper | 2 | mg |
| Iodine | 100 | ug (mcg) |
| Iron | 11 | mg |
| Magnesium | 100 | mg |
| Manganese | 1 | mg |
| Molybdenum | 40 | ug (mcg) |
| Phosphorus | 100 | mg |
| Selenium | 200 | ug (mcg) |
| Zinc | 12 | mg |
| Vitamin A | 640 | ug (mcg) |
| Vitamin C | 60 | mg |
| Vitamin D | 4 | ug (mcg) |
| Vitamin E | 19 | mg |
| Vitamin K | 20 | ug (mcg) |
| Thiamin | 1.1 | mg |
| Riboflavin | 1.4 | mg |
| Niacin | 16 | mg |
| Vitamin B6 | 2 | mg |
| Folic acid | 200 | ug (mcg) |
| Vitamin B12 | 0.8 | ug (mcg) |
| Pantothenic acid | 4 | mg |
| Biotin | 140 | μg (mcg) |

The amounts of nutrients expected to give physiologically similar results are recognized to be + or −15% of the specified value. Thus, the preferred range of nutrient amounts that would give the response as noted in Table 2 is set forth in Table 3 below.

TABLE 3
Preferred range of values for nutritional supplement

| | | |
|---|---|---|
| Calcium | 85-115 | mg |
| Chromium | 51-69 | μ (mcg) |
| Copper | 1.7-2.3 | mg |
| Iodine | 85-115 | μ (mcg) |
| Iron | 9.4-12.6 | mg |
| Magnesium | 85-115 | mg |
| Manganese | 0.85-1.15 | mg |
| Molybdenum | 34-46 | μ (mcg) |
| Phosphorus | 85-115 | mg |
| Selenium | 170-230 | μ (mcg) |
| Zinc | 10.2-13.8 | mg |
| Vitamin A | 544-736 | μ (mcg) |
| Vitamin C | 51-69 | mg |
| Vitamin D | 3.4-4.6 | μ (mcg) |
| Vitamin E | 8.5-11.5 | mg |
| Vitamin K | 17-23 | μ (mcg) |
| Thiamin | 0.94-1.26 | mg |
| Riboflavin | 1.19-1.61 | mg |
| Niacin | 13.6-18.4 | mg |
| Vitamin B6 | 1.7-2.3 | mg |
| Folic acid | 170-230 | μ (mcg) |
| Vitamin B12 | 0.68-0.92 | μ (mcg) |
| Pantothenic acid | 3.4-4.6 | mg |
| Biotin | 120-160 | μg (mcg) |

### Example 3

### Immune Responses and Infection-Related Morbidity

A study was also conducted to compare the immune responses of the control group and for the group receiving the various levels of supplementation. Immune responses were comparable in the two groups at base line. However, the adults given the multinutrient showed a much higher response in all the parameters tested (Table 5) including the number of T lymphocytes, CD4+ helper T cells, lymphocyte response to mitogen PHA, interleukin-2 production by mitogen-stimulated lymphocytes, and antibody production after booster injection of tetanus toxoid.

Infection rate was determined meticulously and showed a significant reduction in the group receiving the multinutrient as shown in Table 4 below.

TABLE 4
Randomized controlled trial of the nutritional supplement for adults.
Immune responses were measured at the end of one year and the incidence
of infection observed for the 12-month duration of the study. Sample size
of 56 subjects per groups, with 1.0 gender ratio in each group.

| | Supplement | Placebo | p |
|---|---|---|---|
| T lymphocytes (%) | 71(2) | 62(2) | <0.05 |
| CD4+ T cells (%) | 52(2) | 40(4) | <0.01 |
| Lymphocyte stimulation | 83(4) | 52(6) | <0.01 |
| Index | | | |
| Interleukin-2 (U/ml) | 17(2) | 8(3) | <0.001 |
| Antibody response | | | |
| To tetanus toxoid (U/ml) | 1.48(0.15) | 0.87(0.34) | <0.001 |
| Infection (days/yr) | 3.4(0.5) | 6.1(0.7) | <0.001 |

Values are shown as median (standard deviation)

The data presented in this document show that the administration daily of a multinutrient designed to meet the unique requirements of adults enhances immune responses and reduces infection in this age group.

The invention being thus described, it will be obvious that the same may be varied in many ways. Such variations are not to be regarded as a departure from the spirit and scope of the invention and all such modifications are intended to be included within the scope of the following claims.

1. A nutritional supplement for adults of 20 to 50 years of age for improving the immunological status of adults, said nutritional supplement comprising:
Chromium in the amount of about 51 to about 69 μg;
Copper in the amount of about 1.7 to about 2.3 mg;
Iodine in the amount of about 85 to about 115 μg;
Iron in the amount of about 9.4 to about 12.6 mg;
Manganese in the amount of about 0.85 to about 1.15 mg;
Molybdenum in the amount of about 34 to about 46 μg;
Phosphorus in the amount of about 85 to about 115 mg;
Selenium in the amount of about 170 to about 230 μg;
Zinc in the amount of about 10.2 to about 13.8 mg;
Vitamin A in the amount of about 544 to about 736 μg;
Vitamin C in the amount of about 51 to about 69 mg;
Vitamin D in the amount of about 3.4 to about 4.6 μg;
Vitamin E in the amount of about 8.5 to about 11.5 mg;
Vitamin K in the amount of about 17 to about 23 μg;
Vitamin B₁ in the amount of about 0.94 to about 1.26 mg;
Vitamin B₂ in the amount of about 1.19 to about 1.61 mg;
Niacin in the amount of about 13.6 to about 18.4 mg;
Vitamin B₆ in the amount of about 1.7 to about 2.3 mg;
Folic acid in the amount of about 170 to about 230 μg;
Vitamin B₁₂ in the amount of about 0.68 to about 0.92 μg;
Pantothenic acid in the amount of about 3.4 to about 4.6 mg; and
Biotin in the amount of about 120 to about 160 μg.

2. The nutritional supplement of claim 1, further comprising calcium in the amount of about 85 to about 115 mg.

3. The nutritional supplement of claim 1, further comprising magnesium in the amount of about 85 to about 115 mg.

4. The nutritional supplement of claim 1, further comprising:
Calcium in the amount of about 85 to about 115 mg; and
Magnesium in the amount of about 85 to about 115 mg.

5. The nutritional supplement of claim 1 wherein said supplement comprises:
Chromium in the amount of about 60 μg;
Copper in the amount of about 2 mg;
Iodine in the amount of about 100 μg;
Iron in the amount of about 11 mg;
Manganese in the amount of about 1 mg;
Molybdenum in the amount of about 40 μg;
Phosphorus in the amount of about 100 mg;
Selenium in the amount of about 200 μg;
Zinc in the amount of about 12 mg;
Vitamin A in the amount of about 640 μg;
Vitamin C in the amount of about 60 mg;
Vitamin D in the amount of about 4 μg;
Vitamin E in the amount of about 19 mg;
Vitamin K in the amount of about 20 μg;
Vitamin B₁ in the amount of about 1.1 mg;
Vitamin B₂ in the amount of about 1.4 mg;
Niacin in the amount of about 16 mg;
Vitamin B₆ in the amount of about 2 mg;
Folic acid in the amount of about 200 μg;
Vitamin B₁₂ in the amount of about 0.8 μg;
Pantothenic acid in the amount of about 4 mg; and
Biotin in the amount of about 140 μg.

6. The nutritional supplement of claim 5, further comprising calcium in the amount of about 100 mg.

7. The nutritional supplement of claim 5, further comprising magnesium in the amount of about 100 mg.

8. The nutritional supplement of claim 5, further comprising:
Calcium in the amount of about 100 mg; and
Magnesium in the amount of about 100 mg.

9. A method for improving the immunological status of adults of 20 to 50 years of age, said method comprising administering a nutritional supplement to an adult, the nutritional supplement comprising:
Chromium in the amount of about 51 to about 69 μg;
Copper in the amount of about 1.7 to about 2.3 mg;
Iodine in the amount of about 85 to about 115 μg;
Iron in the amount of about 9.4 to about 12.6 mg;
Manganese in the amount of about 0.85 to about 1.15 mg;
Molybdenum in the amount of about 34 to about 46 μg;
Phosphorus in the amount of about 85 to about 115 mg;
Selenium in the amount of about 170 to about 230 μg;
Zinc in the amount of about 10.2 to about 13.8 mg;
Vitamin A in the amount of about 544 to about 736 μg;
Vitamin C in the amount of about 51 to about 69 mg;
Vitamin D in the amount of about 3.4 to about 4.6 μg;
Vitamin E in the amount of about 8.5 to about 11.5 mg;
Vitamin K in the amount of about 17 to about 23 μg;
Vitamin B₁ in the amount of about 0.94 to about 1.26 mg;
Vitamin B₂ in the amount of about 1.19 to about 1.61 mg;
Niacin in the amount of about 13.6 to about 18.4 mg;
Vitamin B₆ in the amount of about 1.7 to about 2.3 mg;
Folic acid in the amount of about 170 to about 230 μg;
Vitamin B12 in the amount of about 0.68 to about 0.92 μg;
Pantothenic acid in the amount of about 3.4 to about 4.6 mg; and
Biotin in the amount of about 120 to about 160 μg.

10. The method of claim 9, wherein the nutritional supplement further comprises calcium in the amount of about 85 to about 115 mg.

11. The method of claim 9, wherein the nutritional supplement further comprises magnesium in the amount of about 85 to about 115 mg.

12. The method of claim 9, wherein the nutritional supplement further comprises:
Calcium in the amount of about 85 to about 115 mg; and
Magnesium in the amount of about 85 to about 115 mg.

13. The method of claim 9 wherein said supplement comprises:
Chromium in the amount of about 60 μg;
Copper in the amount of about 2 mg;
Iodine in the amount of about 100 μg;
Iron in the amount of about 11 mg;
Manganese in the amount of about 1 mg;
Molybdenum in the amount of about 40 μg;
Phosphorus in the amount of about 100 mg;
Selenium in the amount of about 200 μg;
Zinc in the amount of about 12 mg;
Vitamin A in the amount of about 640 μg;
Vitamin C in the amount of about 60 mg;
Vitamin D in the amount of about 4 μg;
Vitamin E in the amount of about 19 mg;
Vitamin K in the amount of about 20 μg;
Vitamin B₁ in the amount of about 1.1 mg;
Vitamin B₂ in the amount of about 1.4 mg;
Niacin in the amount of about 16 mg;
Vitamin B₆ in the amount of about 2 mg;
Folic acid in the amount of about 200 μg;
Vitamin B₁₂ in the amount of about 0.8 μg;
Pantothenic acid in the amount of about 4 mg; and
Biotin in the amount of about 140 μg.

14. The method of claim 13, wherein the nutritional supplement further comprises calcium in the amount of about 100 mg.

15. The method of claim 13, wherein the nutritional supplement further comprises magnesium in the amount of about 100 mg.

16. The method of claim 13, wherein the nutritional supplement further comprises:
Calcium in the amount of about 100 mg; and
Magnesium in the amount of about 100 mg.

17. A method for maintaining optimal health of an adult in need thereof, comprising administering the supplement of claim 1.

18. A method for maintaining optimal health of an adult in need thereof, comprising administering the supplement of claim 4.

19. A method for maintaining optimal health of an adult in need thereof, comprising administering the supplement of claim 5.

20. A method for maintaining optimal health of an adult in need thereof, comprising administering the supplement of claim 8.
